(19) United States
(12) Patent Application Publication
(10) Pub. No.: US 2015/0258168 A1
(43) Pub. Date: Sep. 17, 2015

(54) MODULATION OF PODOPLANIN MEDIATED PLATELET ACTIVATION

(71) Applicant: OKLAHOMA MEDICAL RESEARCH FOUNDATION, Oklahoma City, OK (US)

(72) Inventor: Lijun Xia, Oklahoma City, OK (US)

(73) Assignee: OKLAHOMA MEDICAL RESEARCH FOUNDATION, Oklahoma City, OK (US)

(21) Appl. No.: 14/426,182

(22) PCT Filed: Sep. 12, 2013
(86) PCT No.: PCT/US13/59443

§ 371 Date: Mar. 5, 2015

Related U.S. Application Data
(60) Provisional application No. 61/700,097, filed on Sep. 12, 2012.

(51) Int. Cl. A61K 38/17; A61K 31/661; A61K 45/06; C07K 16/28; C12N 15/113; C07K 14/47; C07K 16/18

(52) U.S. Cl. CPC A61K 38/17; C07K 14/47; A61K 31/661; C07K 16/18; C07K 16/2851; C12N 15/113; A61K 45/06; C12N 2310/14; C07K 2317/76

(57) ABSTRACT

The present invention relates generally to the use of modulators of podoplanin (PDPN) mediated platelet activation. For example, an agonist or mimic of podoplanin (PDPN)/C-type lectin-like receptor 2 (CLEC-2) signaling may be used to inhibit vascular leakage or promote vascular integrity. Alternatively, an antagonist of podoplanin (PDPN)/C-type lectin-like receptor 2 (CLEC-2) signaling may be used to inhibit platelet activation.

### PRIORITY CLAIM

This application claims benefit of priority to U.S. Provisional Application Ser. No. 61/700,097, filed Sep. 12, 2012, the entire contents of which are hereby incorporated by reference.

### GOVERNMENT SUPPORT CLAUSE

This invention was made with government support under grant no. P01 HL085607 awarded by the National Institutes of Health. The government has certain rights in the invention.

### BACKGROUND OF THE INVENTION

1. Field of the Invention

The present invention relates generally to the fields of vascular biology, neurology, medicine and pathology. More particularly, it concerns the use of functional modulators of podoplanin (PDPN) in platelet activation, specifically by activation of C-type lectin-like receptor 2 (CLEC-2).

2. Description of Related Art

Vascular integrity in the brain is considered to be primarily developed and maintained by the blood-brain barrier (BBB). Hower, the role that platelet activation and thrombin generation may play in functional vascular integrity in the developing and mature CNS remains unknown. The current model of hemostasis suggests that basic mechanisms of platelet activation and thrombin generation are deployed systemically as a response to vessel injury. Currently, drugs that alter platelet function are effective, but their applications are often limited by complications (e.g., bleeding, thrombosis) since they alter hemostasis in a generalized manner. As such, the ability to provide protection against CNS hemorrhage that does not raise the risk of systemic arterial thrombosis is highly desirable. Thus, the ability to control vascular integrity in vivo both spatially and temporally would provide a means to intervene in bleeding or thrombosis diseases in a tissue-specific manner.

### SUMMARY OF THE INVENTION

Thus, in accordance with the present invention, there is provided a method of inhibiting vascular leakage in central nervous system (CNS) tissue in a subject comprising administering to the subject an agonist or mimic of podoplanin (PDPN)/C-type lectin-like receptor 2 (CLEC-2) signaling. The CNS tissue may be brain tissue. The vascular leakage may be due to trauma, stroke, or inflammation. The agonist or mimic may be administered systemically. The agonist or mimic may be delivered to CNS tissue. The agonist or mimic may be delivered multiple times, including continuously over a period of time exceeding 1 hour. The agonist or mimic may be delivered within 2 hours of the initiation of vascular leakage.

The subject is a human or a non-human mammal. The agonist may be s a soluble form of PDPN. The mimic may be a PDPN mimic. The agonist may be a downstream effector that results from PDPN/CLEC-2 signaling, such as sphingosine-1-phosphate (S1P). The mimic may be a mimic of a downstream effector that results from PDPN/CLEC-2 signaling, such as an S1P receptor 1 (S1PR1) agonist. The method may further comprise administering to the subject a second agent that inhibits vascular leakage. The second agent may be administered at the same time as the agonist or mimic, or administered before or after the agonist or mimic, or administered on an alternating basis with the agonist or mimic.

In another embodiment, there is provided a method of promoting vascular integrity in CNS tissue in a subject comprising administering to the subject an agonist or mimic of PDPN/CLEC-2 signaling. The agonist or mimic may be administered systemically. The agonist or mimic may be delivered to the CNS tissue. The subject may be a human. The agonist may be a soluble form of PDPN. The mimic is a PDPN mimic. The agonist may be a downstream effector that results from PDPN/CLEC-2 signaling such as S1P. The mimic may be a mimic of a downstream effector that results from PDPN/CLEC-2 signaling, such as an S1PR1 agonist.

In yet another embodiment, there is provided a method of inhibiting platelet activation in CNS tissue in a subject comprising administering to the subject an antagonist of PDPN)/CLEC-2 signaling. The CNS tissue may be is brain tissue. The platelet activation may lead to thrombosis or to stroke. The antagonist is administered systemically. The antagonist may be delivered to the CNS tissue. The antagonist may be delivered multiple times. The subject may be a human or a non-human mammal. The antagonist may be an antibody that binds selectively to PDPN or CLEC-2, or an inactive fragment of PDPN or CLEC-2 that interferes with the binding of PDPN to CLEC-2. Alternatively, the antagonist may be a siRNA that inhibits production of PDPN or CLEC-2.

In yet another embodiment, there is provided a method of promoting platelet activation in order to maintain vascular integrity in tissues other than CNS, such as the lung and skin, in a subject comprising administering to the subject an agonist of PDPN/CLEC-2 signaling. The agonist may be a soluble form of PDPN. The mimic is a PDPN mimic. The agonist may be a downstream effector that results from PDPN/CLEC-2 signaling such as SIP. The mimic may be a mimic of a downstream effector that results from PDPN/CLEC-2 signaling, such as an S1PR1 agonist. The subject may suffer from an infection or even sepsis.

In yet another embodiment, there is provided a method of inhibiting platelet activation in tissues other than CNS, such as the lung and skin, in a subject comprising administering to the subject an antagonist of PDPN/CLEC-2 signaling. The platelet activation may lead to thrombosis or to stroke. The antagonist is administered systemically. The antagonist may be delivered to the tissue. The antagonist may be delivered multiple times. The subject may be a human or a non-human mammal. The antagonist may be an antibody that binds selectively to PDPN or CLEC-2, or an inactive fragment of PDPN or CLEC-2 that interferes with the binding of PDPN to CLEC-2. Alternatively, the antagonist may be a siRNA that inhibits production of PDPN or CLEC-2. The mimic may be a mimic of a downstream effector that results from PDPN/CLEC-2 signaling, such as an S1PR1 antagonist.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Pdpn^{-/-} mice develop spontaneous brain hemorrhage. (FIG. 1A) WT and Pdpn^{-/-} brains at embryonic stages. E10.5-12.5 are gross images. E13.5 and H&E-stained sagittal sections of WT and Pdpn^{-/-} brains. E15.5 are gross images of sagittal sections of WT and Pdpn^{-/-} embryo heads. Arrows indicate bleeding. Asterisk marks intraventricular bleeding. (FIG. 1B) Confocal microscopy images of WT and Pdpn^{-/-} brain cryosectins show that PDPN is co-localized with βIII tubulin, a neuronal cell marker, in E12.5 WT brain. CD31 is a marker for vascular endothelium.

FIGS. 2A-B. Mice lacking PDPN in neural cells develop spontaneous brain bleeding. (FIG. 2A) Gross images and H&E-stained sagittal brain sections of P2 WT and NE-Pdpn^{-/-} brains. (FIG. 2B) Cranial view of E12.5 WT and Cre8NE-Pdpn^{-/-} heads. Arrows indicate bleeding.

FIGS. 3A-B. Mice lacking PDPN develop spontaneous hemorrhages in the postnatal brain and PDPN is localized in astrocyte endfeet surrounding larger vessels in the postnatal brain. (FIG. 3A) Gross images and H&E-stained sagittal sections of P1 Pdpn^{-/-} neonatal brains. Arrows indicate bleeding. (FIG. 3B) PDPN is specifically expressed in astrocyte endfeet (Aqp4-positive) surrounding larger vessels (arrows) in WT adult brain. Arrowheads mark smaller vessels without surrounding PDPN.

FIGS. 4A-C. Mice lacking PDPN have defective vascular ultrastructures and impaired vascular integrity in the brain. (FIG. 4A) High resolution confocal images of WT and Pdpn^{-/-} brain vessels and quantifications of vascular diameter. Arrows show abnormal sprouting. (FIG. 4B) Transmission electron microscopy images of WT and Pdpn^{-/-} brain vessels. Arrows indicate tight junctions. Arrowheads mark abnormal interdigitation. Asterisks show abnormal extravascular space. (FIG. 4C) Gross images of cronal sections of WT and NE-Pdpn^{-/-} brain after tMCAO and intravenous injection of Evans blue. Arrows show bleeding. Asterisks shown Evans blue leakages. R, right side (w/tMCAO); L, left side.

FIGS. 5A-B. NE-Pdpn^{-/-} mice are susceptible to bleeding in the brain after tMCAO. (FIG. 5A) Laser Doppler blood perfusion images showing occlusion and reperfusion of blood flow after tMCAO. (FIG. 5B) WT and NE-Pdpn^{-/-} coronal sections. Blue-squared areas were analyzed by H&E staining Black arrows mark bleeding spots. White arrows indicate edema. L, left: R, right side (w/tMCAO).

FIG. 6. NE-Pdpn^{-/-} adult brain exhibits bleeding from larger vessels after tMCAO. Confocal imaging show that PDPN is specifically around larger vessels (arrows) but not small vessels (arrowheads) in the WT brain. NE-Pdpn^{-/-} vessels have no PDPN, confirming the deletion. Bleeding (Ter119, red cell marker) is specifically associated with larger vessels (arrow) in the NE-Pdpn^{-/-} brain. Insets show red cells are inside PDPN-positive WT vessels, but outside NE-Pdpn^{-/-} vessels. Lectin (tomato) highlights vascular endothelium.

FIGS. 7A-B. Clec-2^{-/-} mice develop spontaneous brain hemorrhage. (FIG. 7A) Gross images of WT and Clec-2^{-/-} embryos. E12.5 are sagittal sections of embryos. Arrows indicate bleeding. Arrowheads mark abnormal blood-filled lymphatic vessels. (FIG. 7B) Gross images of H&E-stained sagittal sections of WT and Clec-2^{-/-} neonates. Arrows mark bleeding.

FIGS. 8A-C. Endogenous CLEC-2 is expressed only on platelets, but not on Gr1⁺ neutrophils and monocytes and embryonic loss of platelets results in CNS hemorrhage. (FIGS. 8A-B) WT platelets (FIG. 8A) and WT or PSGL-1^{-/-} peripheral leukocytes (FIG. 8B) were stained with antibodies to murine CLEC-2 (17D9, rat IgG2b) and Gr1 (myeloid cell marker) with or without 5 mM EDTA. (FIG. 8C) H&E-stained coronal embryonic brain sections of E12.5 PF4-Cre;R26R-DTA and control R26R-DTA embryos. CNS hemorrhages (arrows) were observed in PF4-Cre;R26R-DTA mice lacking platelets.

FIG. 9. Activation of CLEC-2 induces release of S1P from platelets. S1P in supernatants of WT and Clec-2^{-/-} platelets after incubation with anti-CLEC-2 antibody INU1 (INU1-treated) or isotype control (sham-treated) was measured by ELISA. S1P in platelet lysates (plt lysates) were measured as controls. Data represent the mean±SD (n=4) from two experiments.

FIGS. 10A-B. Generation of Clec-2 conditional mouse line. (FIG. 10A) Targeting strategy for generating a conditional Clec-2 allele. Delection of exons 3 and 4 results in a frameshift and premature STOP in all downstream splice events and blocks expression of CLEC-2 extracellular domain. (FIG. 10B) Targeted W4 ES cells (129 SvEv, agouti) were injected into albino blastocytes, so agouti color of the chimeras represents contribution from ES cells. Arrows indicate chimeras recently obtained with almost 100% chimerism.

FIG. 11. Detection of sphingosine (18:1), sphingosine-1-phosphate (17:1), and sphingosine-1-phosphate (18:1) by capillary column LC-tandem mass spectrometry.

FIG. 12. S1PR1 agonist SEW2871 ameliorates brain bleeding in NE-Pdpn^{-/-} brain after tMCAO. Gross images of cronal sections of NE− Pdpn^{-/-} brains. The mice (6-wks-old) were treated either with SEW2871 or DMSO (solvent of SEW2871) immediately after reperfusion. 24 hrs after the reperfusion, mice were intravenously injected with Evans blue, sacrificed and immediately perfused with saline and 4% PFA. Arrows show bleeding. Asterisks show leakage of Evans blue. R, right side (tMCAO side).

FIGS. 13A-C. Working model illustrating PDPN-CLEC-2-mediated platelet activation leads to vascular integrity. (FIG. 13A) WT vessel without injury. (FIG. 13B) After injury, S1P released from PDPN-CLEC-2-activated platelets results in an increase in local concentration of S1P, which acts on its receptor S1PR1, to protect vascular integrity from bleeding. (FIG. 13C) Lack of PDPN or CLEC-2 fails to activate platelets to release S1P and results in impaired vascular integrity and bleeding. EC, endothelial cell; MC, mural cell; NC, neuronal cell or astrocyte; Plt, platelet; TJ, tight junction.

FIGS. 14A-B. Mice lacking S1PR1 are susceptible to vascular leakage during inflammation. (FIG. 14A) Gross images of skins of WT and S1PR1-deficient mice. The mice (8-wks-old) were immune challenged with reverse Arthus reaction. One hour before sacrifice, the mice were intravenously injected with Evans blue to detect the vascular leakage. After killing, mice were immediately perfused with saline and 4% PFA. Arrows show leakage of Evans blue. (FIG. 14B) Concentration of Evans blue in skin tissues 4 hrs after immune challenging. Comparing with WT mice, S1PR1 deficient mice had significant higher concentration of Evans blue, indicating an increased vascular leakage.

### DESCRIPTION OF ILLUSTRATIVE EMBODIMENTS

Starting around embryonic day 10 (E10) in mice, endothelial cells from the perineural vascular plexus begin actively invading the rapidly growing neural tissue (Eichmann et al., 2005; Bautch and James, 2009; Tam and Watts, 2010). The nascent vasculatures are prone to bleeding. Postnatally the brain exists in a confined space in which a small amount of extravasated blood rapidly causes neurologic deficits. To protect vascular integrity the brain develops and maintains the blood-brain barrier (BBB) (Jain, 2003; Murakami and Simons, 2009). However, BBB is not fully formed during embryonic development especially before E14 in mice, and larger vessels such as arterioles have incomplete BBB properties in the postnatal brain (Daneman et al., 2010; Abbott et al., 2006). The mechanisms protecting vascular integrity both spatially and temporally where BBB is reduced are unclear. The brain vascular integrity is also protected by hemostasis such as platelet activation. However, in the current paradigm for hemostasis the mechanisms of platelet activation occur systemically and hemostatic responses occur similarly in all tissues (Furie and Furie, 2008).

Recent reports show an important new mechanism of platelet activation that relies on activation of the platelet CLEC-2 receptor (Bertozzi et al., 2010; May et al., 2009; Severin et al., 2011). The inventor has confirmed these findings of these studies and, further, found in that O-glycoprotein podoplanin (PDPN) is an important mediator of this mechanism. It is highly expressed in neural cells surrounding vessels in the developing CNS and is localized in astrocyte endfeet surrounding larger vessels in the postnatal brain. Significantly, mice with global deficiency of PDPN (Pdpn^{-/-}) or of CLEC-2 (Clec-2^{-/-}) develop identical CNS-specific hemorrhages primarily during E10.5-14.5, supporting that PDPN and CLEC-2 act as partners for this function. Furthermore, mice lacking PDPN in astrocytes develop large and focal brain bleeding after injury, suggesting breached larger vessels, such as arterioles. These results support that the interaction between PDPN on neural cells and CLEC-2 on platelets is important for vascular integrity in the developing and developed brain when and where BBB is reduced. The inventor proposes the use of pharmacological intervention to modulate the function of the PDPN-CLEC-2 interaction in vascular integrity in the CNS. Identification of such a brain-specific activation of platelets provides a novel mechanism for the affecting maintenance of vascular integrity in CNS, which is vulnerable to hemorrhage, as well as regulating pathologic platelet aggregation. Such findings significantly alter the current paradigm for hemostasis, provide new insights into the pathogenesis of CNS hemorrhage, and set the foundation for new tissue-specific therapies for important CNS diseases such as stroke.

### I. Fluid and Hemodynamic Derangements

A. Platelets and Hemostasis

Blood coagulation and platelet aggregation are two basic hemostatic mechanisms. Platelets are rich in secretory vesicles such as dense granules, α-granules, and lysosomes. Under physiological conditions, circulating platelets do not interact with the vessel walls. In response to vascular injury, platelets are activated by the interaction of platelet integrins and glycoprotein VI (GPVI) with exposed underlying extracellular matrix (ECM) such as collagen to form aggregates that seal injured vessels. Platelet activation also leads to secretion of granule substances, such as ADP and thromboxane A₂ to reinforce platelet aggregation, and of procoagulant molecules to effectively form a platelet-fibrin plug or thrombus that ensures vascular integrity after wounding. Aside from their role in thrombosis and hemostasis, substances released from activated platelets have complex biological functions such as those in inflammation, wound healing, and angiogenesis. Recent studies show that activated platelets are critical in preventing hemorrhage from newly formed vessels in tumor models, and protect mature brain vessels from bleeding under inflammation. However, the mechanisms by which platelets are activated and how platelets function in these pathological processes are unknown.

B. Tissue-Specific Hemostasis and Vascular Integrity in the CNS

In the current paradigm for hemostasis, mechanisms of platelet activation occur systemically, and hemostatic responses occur similarly in all tissues. The studies by the inventor and others conclude that a novel, potentially tissue-specific mechanism of platelet activation exists in which PDPN activates the platelet CLEC-2 receptor. The inventor observed that loss of CLEC-2 or PDPN results in hemorrhage that occurs specifically in the developing brain. They have determined the role of the PDPN-CLEC-2 pathway in CNS tissue-specific activation of platelets. Identification of a direct mechanism for tissue-dependent activation of platelets will help resolve a novel tissue-specific hemostatic mechanism for the development and maintenance of vascular integrity in CNS, an organ vulnerable to hemorrhage. Such findings would significantly alter the paradigm for hemostasis, provide new insights into the pathogenesis of CNS hemorrhage, and set the foundation for new tissue-specific therapeutic strategies to treat these diseases.

C. Thrombosis Disorders

Thrombotic events involved the formation of a blood clot inside a blood vessel, obstructing the flow of blood through the circulatory system. When a blood vessel is injured, the body uses platelets (thrombocytes) and fibrin to form a blood clot to prevent blood loss. Alternatively, even when a blood vessel is not injured, blood clots may form in the body if the proper conditions present themselves. If the clotting is too severe and the clot breaks free, the traveling clot is now known as an embolus. Thromboembolism is the combination of thrombosis and its main complication, embolism.

A stroke, or cerebrovascular accident (CVA), is the rapid loss of brain function(s) due to disturbance in the blood supply to the brain. This can be due to ischemia (lack of blood flow) caused by blockage (thrombosis, arterial embolism), or a hemorrhage (leakage of blood). As a result, the affected area of the brain cannot function, which might result in an inability to move one or more limbs on one side of the body, inability to understand or formulate speech, or an inability to see one side of the visual field.

A stroke is a medical emergency and can cause permanent neurological damage, complications, and death. It is the leading cause of adult disability in the United States and Europe and the second leading cause of death worldwide. Risk factors for stroke include old age, hypertension (high blood pressure), previous stroke or transient ischemic attack (TIA), diabetes, high cholesterol, cigarette smoking and atrial fibrillation. High blood pressure is the most important modifiable risk factor of stroke.

An ischemic stroke is occasionally treated in a hospital with thrombolysis (also known as a “clot buster”), and some hemorrhagic strokes benefit from neurosurgery. Treatment to recover any lost function is termed stroke rehabilitation, ideally in a stroke unit and involving health professions such as speech and language therapy, physical therapy and occupational therapy. Prevention of recurrence may involve the administration of antiplatelet drugs such as aspirin and dipyridamole, control and reduction of hypertension, and the use of statins. Selected patients may benefit from carotid endarterectomy and the use of anticoagulants.

In thrombotic stroke a thrombus (blood clot) usually forms around atherosclerotic plaques. Since blockage of the artery is gradual, onset of symptomatic thrombotic strokes is slower. A thrombus itself (even if non-occluding) can lead to an embolic stroke (see below) if the thrombus breaks off, at which point it is called an “embolus.” Two types of thrombosis can cause stroke:
Large vessel disease involves the common and internal carotids, vertebral, and the Circle of Willis. Diseases that may form thrombi in the large vessels include (in descending incidence): atherosclerosis, vasoconstriction (tightening of the artery), aortic, carotid or vertebral artery dissection, various inflammatory diseases of the blood vessel wall (Takayasu arteritis, giant cell arteritis, vasculitis), noninflammatory vasculopathy, Moyamoya disease and fibromuscular dysplasia.
Small vessel disease involves the smaller arteries inside the brain: branches of the circle of Willis, middle cerebral artery, stem, and arteries arising from the distal vertebral and basilar artery. Diseases that may form thrombi in the small vessels include (in descending incidence): lipohyalinosis (build-up of fatty hyaline matter in the blood vessel as a result of high blood pressure and aging) and fibrinoid degeneration (stroke involving these vessels are known as lacunar infarcts) and microatheroma (small atherosclerotic plaques).
   Sickle-cell anemia, which can cause blood cells to clump up and block blood vessels, can also lead to stroke. A stroke is the second leading killer of people under 20 who suffer from sickle-cell anemia.

An embolic stroke refers to the blockage of an artery by an arterial embolus, a travelling particle or debris in the arterial bloodstream originating from elsewhere. An embolus is most frequently a thrombus, but it can also be a number of other substances including fat (e.g., from bone marrow in a broken bone), air, cancer cells or clumps of bacteria (usually from infectious endocarditis). Because an embolus arises from elsewhere, local therapy solves the problem only temporarily. Thus, the source of the embolus must be identified. Because the embolic blockage is sudden in onset, symptoms usually are maximal at start. Also, symptoms may be transient as the embolus is partially resorbed and moves to a different location or dissipates altogether. Emboli most commonly arise from the heart (especially in atrial fibrillation) but may originate from elsewhere in the arterial tree. In paradoxical embolism, a deep vein thrombosis embolises through an atrial or ventricular septal defect in the heart into the brain.

Cardiac causes can be distinguished between high and low-risk:
High risk: atrial fibrillation and paroxysmal atrial fibrillation, rheumatic disease of the mitral or aortic valve disease, artificial heart valves, known cardiac thrombus of the atrium or ventricle, sick sinus syndrome, sustained atrial flutter, recent myocardial infarction, chronic myocardial infarction together with ejection fraction <28 percent, symptomatic congestive heart failure with ejection fraction <30 percent, dilated cardiomyopathy, Libman-Sacks endocarditis, Marantic endocarditis, infective endocarditis, papillary fibroelastoma, left atrial myxoma and coronary artery bypass graft (CABG) surgery.
Low risk/potential: calcification of the annulus (ring) of the mitral valve, patent foramen ovale (PFO), atrial septal aneurysm, atrial septal aneurysm with patent foramen ovale, left ventricular aneurysm without thrombus, isolated left atrial “smoke” on echocardiography (no mitral stenosis or atrial fibrillation), complex atheroma in the ascending aorta or proximal arch.
   Cerebral venous sinus thrombosis leads to stroke due to locally increased venous pressure, which exceeds the pressure generated by the arteries. Infarcts are more likely to undergo hemorrhagic transformation (leaking of blood into the damaged area) than other types of ischemic stroke.

It generally occurs in small arteries or arterioles and is commonly due to hypertension, intracranial vascular malformations (including cavernous angiomas or arteriovenous malformations), cerebral amyloid angiopathy, or infarcts into which secondary haemorrhage has occurred. Other potential causes are trauma, bleeding disorders, amyloid angiopathy, illicit drug use (e.g., amphetamines or cocaine). The hematoma enlarges until pressure from surrounding tissue limits its growth, or until it decompresses by emptying into the ventricular system, CSF or the pial surface. A third of intracerebral bleed is into the brain's ventricles. ICH has a mortality rate of 44% after 30 days, higher than ischemic stroke or subarachnoid hemorrhage (which technically may also be classified as a type of stroke).

D. Disorders of Vascular Leakage

Physical trauma is a serious and body-altering physical injury, such as the removal of a limb. Blunt force trauma, a type of physical trauma caused by impact or other force applied from or with a blunt object, whereas penetrating trauma is a type of physical trauma in which the skin or tissues are pierced by an object. Trauma can also be described as both unplanned, such as an accident, or planned, in the case of surgery. Both can be characterized by mild to severe tissue damage, blood loss and/or shock, and both may lead to subsequent infection, including sepsis.

Traumatic hemorrhage accounts for much of the wide ranging international impact of injury, causing a large proportion of deaths and creating great morbidity in the injured. Despite differences in pre-hospital care, the acute management of traumatic hemorrhage is similar around the world and follows well accepted published guidelines. A critically injured patient's care occurs as four, often overlapping segments: the resuscitative, operative, and critical care phases. The diagnosis and control of bleeding should be a high priority during all of the phases of trauma care and is especially important in the patient who is in hemorrhagic shock. Early attempts at hemorrhage control include direct control of visible sources of severe bleeding with direct pressure, pressure dressings, or tourniquets; stabilization of long bone and pelvic fractures; and keeping the patient warm. During the resuscitative phase, warmed intravenous fluids, hypotensive resuscitation prior to surgical control of hemorrhage, and appropriate transfusion of blood and blood products are provided. In the operative phase, surgical control of the hemorrhage and any other injury, and additional transfusion is provided. Finally, the critical care phase provides for post-operative support and tissue perfusion.

Capillary leak syndrome (usually Systemic Capillary Leak Syndrome, SCLS or Clarkson's Disease) is a rare medical condition characterized by self-reversing episodes during which the endothelial cells which line the capillaries are thought to separate for a few days, allowing for a leakage of fluid from the circulatory system to the interstitial space, resulting in a dangerous hypotension (low blood pressure), hemoconcentration, and hypoalbuminemia. It is a life-threatening illness because each episode has the potential to cause damage to, or the failure of, vital organs due to limited perfusion. It is often misdiagnosed as polycythemia, polycythemia vera or sepsis.

E. Disorders of Pathologic Platelet Activation

There are a variety of diseases relating to platelet activation. Thrombocytopenic disorders include hereditary, immune-mediated, and drug-induced thrombocytopenias. Others involve deficiencies of von Willebrand factor-cleaving protease. Still others include disorders of platelet function, such as adhesive protein receptors, platelet secretion, and signal transduction. Other disease states include disseminated intravascular coagulation, coagulation relating to bacterial, viral, and parasitic infection, renal and tumor function defects, as well as coagulation due to allergic and inflammatory diseases, embryonic development, psychiatric and neurologic disorders, and inflammatory bowel disease.

von Willebrand disease (vWD) is the most common inherited bleeding disorder. It is autosomal dominant, and its prevalence is estimated to be as high as 1 case per 1000 population. vWf has a major role in primary hemostasis as mediator of the initial shear-stress-induced interaction of the platelet to the subendothelium via the GP Ib complex. In addition, von Willebrand protein acts as a carrier and stabilizer of coagulation factor VIII by forming a complex in the circulation. In the absence of vWf, the factor VIII activity level is low. In classic hemophilia A, the factor VIII activity level is low because of a defect in factor VIII itself, whereas in von Willebrand disease, the factor VIII activity level is low because of a deficiency in its carrier protein.

Although the common form of von Willebrand disease (type I) results from a quantitative deficiency of vWf, the variants result from abnormalities in the von Willebrand protein. A common variant (type IIA) of von Willebrand disease results from functionally defective vWf that is unable to form multimers or be more susceptible to cleavage by ADAMTS13. In the type IIB variant, the von Willebrand protein has heightened interaction with platelets, even in the absence of stimulation. Platelets internalize these multimers, leading to a deficiency of von Willebrand protein in the plasma. A disorder of platelet GP Ib has also been described. In this condition, increased affinity for von Willebrand protein in the resting stage leads to the deletion of plasma von Willebrand protein. Type III von Willebrand disease is a severe form of von Willebrand disease that is characterized by very low levels of vWf and clinical features similar to hemophilia A, but with autosomal recessive inheritance.

Bernard-Soulier syndrome results from a deficiency of platelet glycoprotein protein Ib, which mediates the initial interaction of platelets with the subendothelial components via the von Willebrand protein. Glanzmann thrombasthenia results from a deficiency of the GP IIb/IIIa complex. Both Bernard-Soulier syndrome and Glanzmann thrombasthenia are characterized by lifelong bleeding. Although platelet transfusions are effective, they should be used only for severe bleeding and emergencies, because alloantibodies often develop in these patients.

### II. Podoplanin, C-Type Lectin-Like Receptor 2 and Antagonists/Agonists Thereof

A. PDPN

PDPN (a.k.a. T1 alpha, gp38 and Aggrus) is a heavily O-glycosylated type I transmembrane protein that is highly expressed in several cell types including neural cells, glomerular podocytes, lymphatic endothelial cells, and some tumor cells. The physiological function of this protein may be related to its mucin-type character. The inventor observed in experiments in vivo that loss of O-glycans in endothelial cells causes impaired expression of PDPN and misconnections between blood and lymphatic vessels during development. The inventor demonstrated in further studies that mice with global deficiency of Pdpn (Pdpn^{-/-}) display a similar phenotype, confirming the requirement for PDPN in the separation of lymphatic vessel from blood vessel during development. The role of PDPN in tissues other than lymphatic vessels is unexplored. Reports suggest that PDPN is highly expressed in the neural tubes of mice as early as E6.5, and that PDPN expression is in almost all nestin-positive neural cells during embryonic development (17). No genetic studies regarding the role of PDPN in the CNS have been reported. The inventor recently found in a preliminary study that Pdpn^{-/-} mice exhibit brain bleeding during embryonic and postnatal development. This finding suggests that PDPN is required for blood vascular integrity in the developing and mature CNS.

The human mRNA is found at accession no. NM_{—}001006624.1 and the mouse mRNA at accession no. NM_{—}010329.2. The human protein is found at accession no. NP_{—}001006625.1, and the mouse protein at accession no. NP_{—}034459.2.

B. CLEC-2

CLEC-2 (aka CLEC1B) is one of a family of type II transmembrane receptors with C-type lectin-like extracellular domains. These domains allow it to bind to glycan ligands. CLEC-2 activates platelets through the non-receptor tyrosine kinase SYK and SLP-76. CLEC-2 is highly and selectively expressed on platelets, although it has also been reported to be expressed on myeloid cells. CLEC-2 was first identified as the receptor responsible for platelet activation by the snake venom rhodocytin. A more biologically significant insight came when CLEC-2 was identified as the receptor responsible for platelet activation by PDPN expressed on tumor cells. The biological roles of CLEC-2 are not understood. The inventor has recently determined that interactions between PDPN on lymphatic endothelial cells and platelet CLEC-2 activates SYK/SLP-76 signaling in platelets, causing platelet aggregation that seals initial embryonic blood-lymphatic vascular connections at E12-13. C/ec-2-deficient mice (Clec-2^{-/-}) display a lymphatic vascular phenotype identical to mice lacking PDPN and the CLEC-2 signaling effectors SYK, SLP-76 and PLCγ2. These studies clearly establish that PDPN is a physiologically relevant ligand for CLEC-2, and reveals a novel platelet function in lymphatic vascular development. Whether PDPN-CLEC-2-mediated platelet activation plays a role other than controlling the separation of blood and lymphatic vessels is unknown. The inventor's recent studies support that PDPN expression in the CNS is required to activate platelet CLEC-2 receptors, serving as a tissue-specific hemostatic pathway that controls the development and maintenance of vascular integrity specifically in the CNS.

C. Antagonists

A variety of molecules may be contemplated as antagonists of PDPN-CLEC-2 including antibodies to either molecule, siRNA's that inhibit production of either molecule, or inactive fragments of either molecule that interfere with the binding of PDPN to CLEC-2 to the binding of PDPN-CLEC-2 to a third molecule.

D. Agonists

Agonists of the PDPN-CLEC-2 interaction may be mimics of these molecules, or a downstream effector that results from PDPN/CLEC-2 signaling. On such downstream effector is SIP, and thus another agonist may thus be an S1PR1 agonist other than SIP.

### III. Methods of Treatment

A. Pharmaceutical Formulations

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when the agent are introduced into a patient. Aqueous compositions of the present invention comprise an effective amount of the agent dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative, a flavoring agent and/or a coloring agent. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

B. Administration

Generally, agents for use in accordance with the present invention will be administered intravenously, i.e., systemically, but may be administered more or less locally, i.e., to the vasculature of the relevant region, such as a site of vascular leakage, or a site of clotting. Administration may be by continuous infusion, for example, using a portable pump, or by a series of bolus injections. Administration may be discontinued and restarted if side effects occur. The drug can be given via a catheter directly placed in the relevant artery or vein.

C. Combined Therapy

In another embodiment, it is envisioned to use agents of the present invention with other therapeutic modalities. Thus, in addition to the therapies described above, one may also provide to the patient more “standard” pharmaceutical therapies. For example, if the goal is hemostasis, one has a variety of options. Chemical/topical agents are often used in surgery settings to stop bleeding. Microfibriller collagen is the most popular choice among surgeons because it attracts the patient's natural platelets and starts the blood clotting process when it comes in contact with the platelets. This topical agent requires normal hemostatic pathway to be properly functional. Direct pressure or pressure dressings are most commonly used in situations where proper medical attention is not available. Putting pressure and/or dressing to a bleeding wound only slows the process of blood loss, allowing for more time to get to an emergency medical setting. Soldiers use this skill during combat when someone has been injured because this process allows for blood loss to be decreased, giving the system time to start coagulation. Sutures and ties are often used to close an open wound, allowing for the injured area to stay free of pathogens and other unwanted debris to enter the site; however, it is also essential to the process of hemostasis. Sutures and ties allow for skin to be joined back together allowing for platelets to start the process of hemostasis at a quicker pace. Using sutures results in a quicker recovery period because the surface area of the wound has been decreased. Physical agents (e.g., gelatin sponge) have been indicated as great hemostatic devices. Once applied to a bleeding area, a gelatin sponge quickly stops or reduces the amount of bleeding present. These physical agents are mostly used in surgical settings as well as after surgery treatments. These sponges absorb blood, allow for coagulation to occur faster, and give off chemical responses that increase the time it takes for the hemostasis pathway to start.

Combinations may be achieved by treating a subject with a single composition or pharmacological formulation that includes both agents, or by contacting the cell with two distinct compositions or formulations, at the same time, wherein one composition includes the expression construct and the other includes the agent. Alternatively, the therapy using AR agonists or stimulated EPCs may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of less than about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either AR agonists or stimulated EPCs, or the other agent will be desired. In this regard, various combinations may be employed. By way of illustration, where AR agonists or stimulated EPCs is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in conjunction with therapies of the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, an antianginal agent, an antibacterial agent or a combination thereof. The following are exemplary of such combinations.

1. Antithrombotic/Fibrinolytic Agents

In certain embodiments, administration of an agent that aids in the removal or prevention of blood clots may be combined with administration of a modulator, particularly in treatment of atherosclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

2. Blood Coagulants

In certain embodiments wherein a patient is suffering from a hemorrhage or an increased likelihood of hemorrhaging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

### IV. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Results

Pdpn^{-/-} embryos develop spontaneous hemorrhages specifically in the CNS during early embryonic development. PDPN is a heavily O-glycosylated transmembrane protein that is highly expressed in several cell types including neural cells, glomerular podocytes, lymphatic endothelial cells, and some tumor cells (Schacht et al., 2003; Ramierez et al., 2003; Kato et al., 2003; Borok et al., 1998). In a previous study to determine the function of O-glycans in endothelial cells, the inventor found that mice lacking endothelial 0-glycans have impaired expression of PDPN and misconnections between blood and lymphatic vessels (Fu et al., 2008). The inventor demonstrated that Pdpn^{-/-} mice display a similar phenotype, uncovering the requirement for PDPN in the separation of lymphatic vessels from blood vessels during development (Fu et al., 2008).

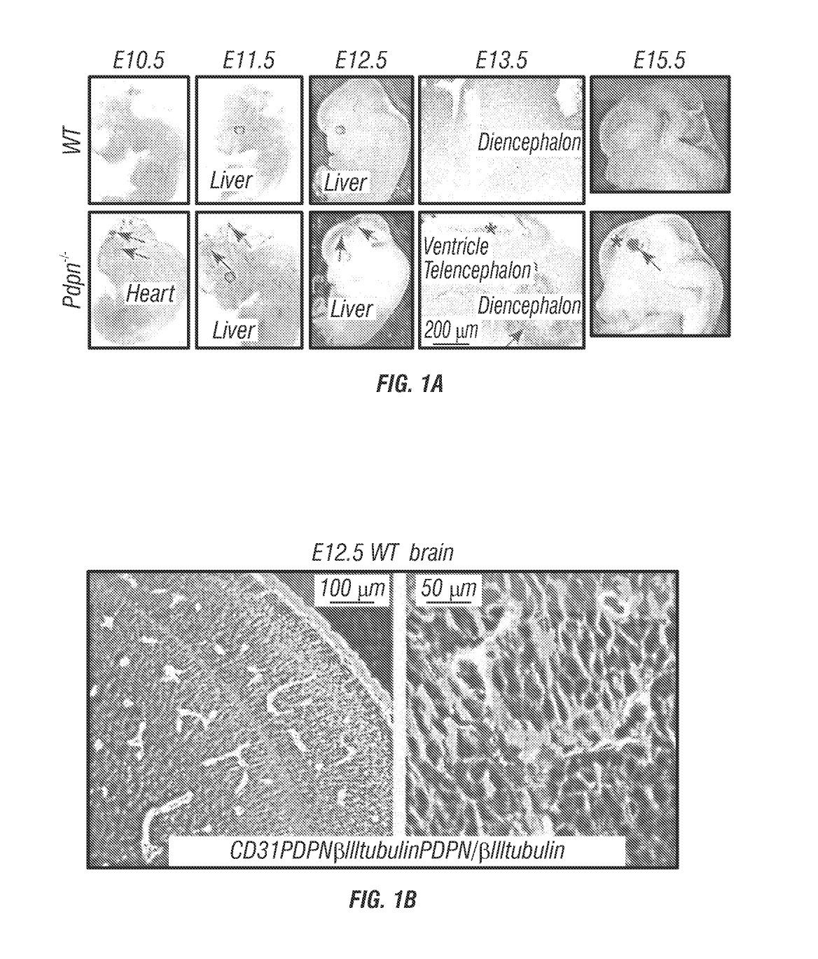

While studying the role of PDPN in lymphatic development in mouse embryos, the inventor discovered that Pdpn^{-/-} embryos exhibited an unreported hemorrhage phenotype specifically in the CNS starting from E10.5 (FIG. 1A). The brain hemorrhages in Pdpn^{-/-} embryos arise spontaneously, are fully penetrant, and occur predominantly between E10.5-14.5 (n=8-20 embryos/stage) (FIG. 1A) when angiogenesis is active and BBB is not formed, suggesting PDPN is required for vascular integrity during this stage. Published reports show that PDPN is expressed in neuroepithelium starting at as early as E6.5 (Kotani et al., 2003). This analysis shows that PDPN is widely expressed in the wild-type (WT) E12.5 brain, especially in cerebral cortex, hippocampus, and thalamus, where bleeding is most prevalent (FIG. 1B, and data not shown). Confocal imaging analyses show that PDPN is expressed in neural cells that are positive for nestin, which is expressed by neuronal cells and glial cell precusors. These PDPN-positive cells also express βIII tubulin (FIG. 1B), a marker for neuronal cells (Kotani et al., 2003), suggesting that most neural cells at this stage are neuronal cells. Significantly, PDPN is highly expressed in neuronal cells surrounding vessels in the early embryonic brain (FIG. 1B). These preliminary studies support the hypothesis that PDPN in neural cells is required for vascular integrity in a CNS-specific manner and that it performs important protective functions before BBB is fully formed and therefore vulnerable to leakage.

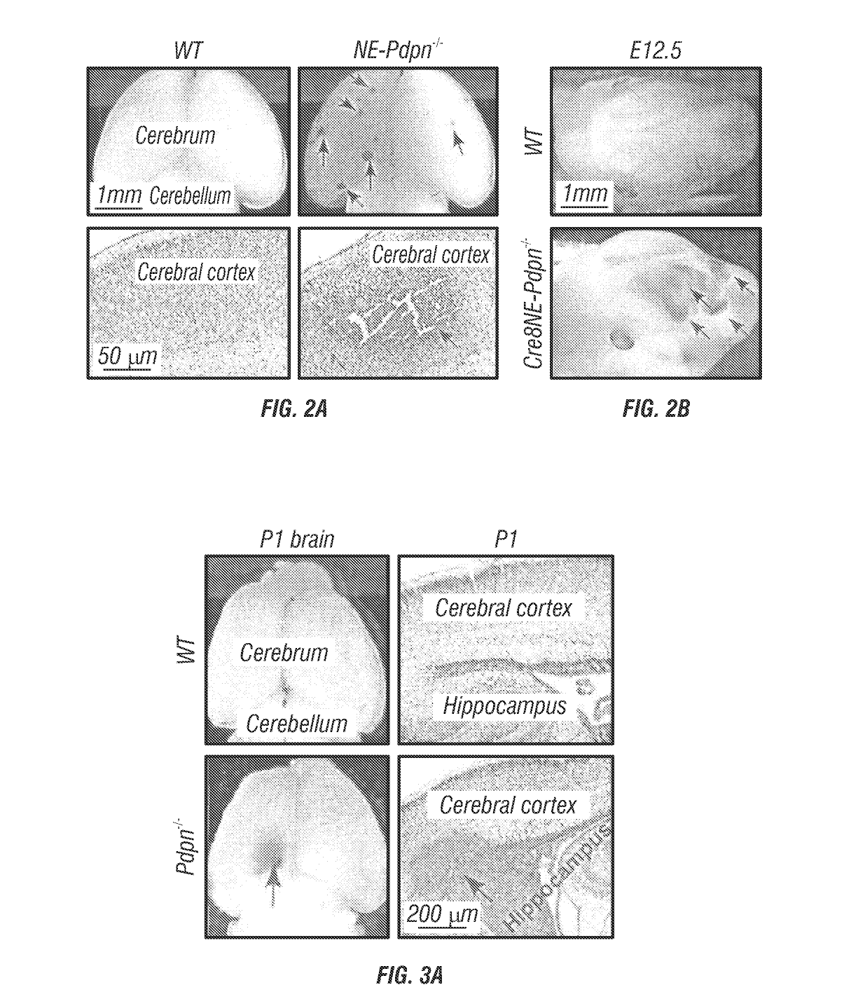

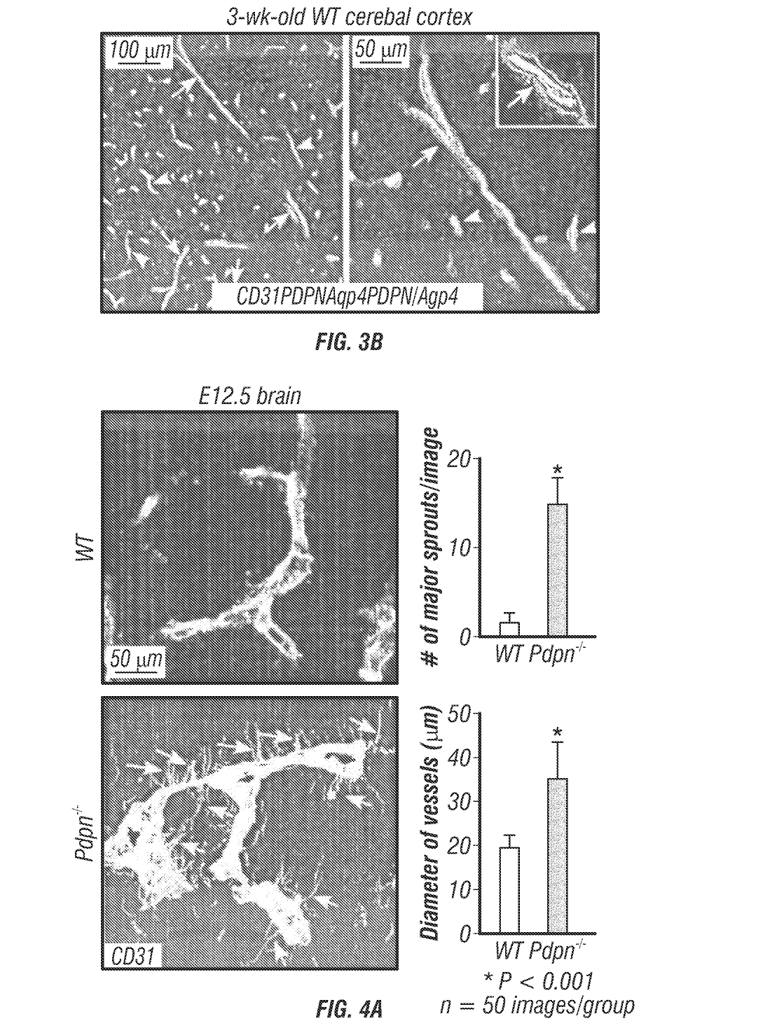

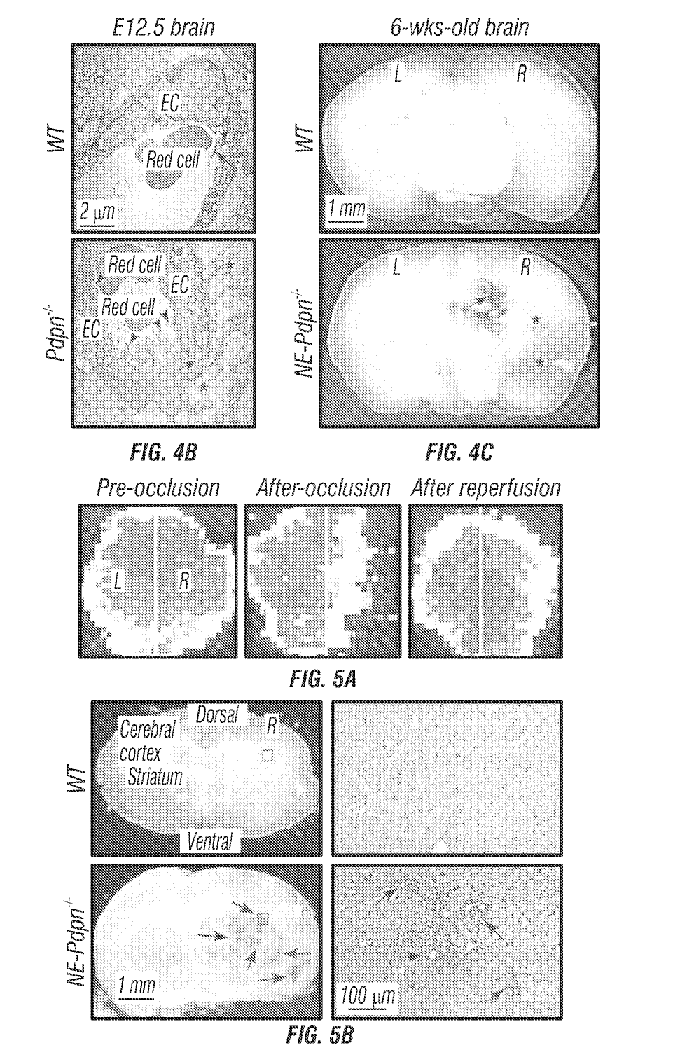

Mice lacking PDPN in neural cells develop spontaneous bleeding. To determine whether PDPN in neural cells is required for integrity of newly formed vessels in the CNS, the inventor generated a novel mouse line in which the major coding exon of the Pdpn gene, exon 2, is flanked by loxP sites (Pdpnf/f, data not shown). He then generated mice lacking PDPN in neural cells (NE-Pdpn^{-/-}) by breeding Pdpnf/f with a nestin-Cre line (JAX), which mediates deletion of the floxed gene at E11 (Tronche et al., 1999). Analysis demonstrated that NE-Pdpn^{-/-} mice that had cerebral hemorrhages primarily after birth (P0-3, n=23 for WT and 25 for KO) (FIG. 2A). This phenotype is likely caused by traumas during delivery, which suggests an important role for PDPN in neural cells in protecting brain vascular integrity from injury. However, in contrast to Pdpn^{-/-} embryos that begin bleeding in the CNS at E10.5, NE-Pdpn^{-/-} embryos displayed no CNS bleeding during E10.5-15.5 (n=30).

Absence of an early embryonic hemorrhagic phenotype can be attributed to residual PDPN mRNA and/or protein produced before deletion of PDPN by Nes-Cre. This is plausible because expression of this Cre transgene begins at E11 after PDPN has already begun to be expressed. To test this, the inventor bred Pdpnf/f with a different nestin-Cre line (Nes-Cre8, from Dr. Weimin Zhong, Yale University), which expresses Cre beginning at E8.5 (Petersen et al., 2002). This allowed the inventor to generate a mouse line in which PDPN is deleted in neural cells at E8.5 (Cre8NEPdpn^{-/-}), prior to CNS bleeding. These studies show that Cre8NE-Pdpn^{-/-} embryos develop brain bleeding at E12.5, reminiscent of Pdpn^{-/-} mice (FIG. 2B). This indicates that PDPN in neural cells is critical for vascular integrity during embryonic development.

PDPN is required for vascular integrity in the postnatal brain. Most Pdpn^{-/-} mice die shortly after birth presumably due to lung failure (Ramirez et al., 2003), but those that survive for a few days exhibit brain hemorrhages (FIG. 3A). This implicates the PDPN is not only important during development but also as an important factor in the postnatal maintenance of CNS vascular integrity. Interestingly, hemorrhages in Pdpn^{-/-} postnatal brains are large and focal, suggesting breached high-pressure, high-volume vessels, such as arterioles. Consistent with this, the inventor found that expression of PDPN becomes localized to astrocyte endfeet that specifically surround large vessels but not capillaries in the postnatal brain (FIG. 3B). Astrocytes are fully differentiated after birth, extend their endfeet to enclose blood vessels and contribute to BBB maturation (Daneman et al., 2010; Abbott et al., 2006; Armulik et al., 2010). Significantly, both NE-Pdpn^{-/-} postnatal brains develop massive hemorrhages (FIG. 2A). These results suggest that PDPN in astrocytes is required to protect the integrity of large vessels in the postnatal brain.

Pdpn^{-/-} mice have abnormal vessel structures in the developing CNS and impaired vascular integrity in the adult brain. High-resolution confocal analysis showed that vessels in the Pdpn−/− embryonic brain were irregular, dilated, and showed increased sprouting relative to the WT controls (FIG. 4A). The inventor used electron microscopy to better characterize the structures of E12.5 Pdpn^{-/-} brain vessels. Pdpn^{-/-} vascular endothelial cells exhibited distorted shapes with dramatically increased abnormal interdigitations relative to WT vessels (FIG. 4B). Endothelial cell tight junctions were weakly present in Pdpn^{-/-} vessels. Many Pdpn^{-/-} vessels were also surrounded by large spaces that appeared to be filled with plasma, suggesting unstable interactions with the extracellular matrix or neighboring cells and accompanying vascular leakage. These results support that vascular integrity is impaired in the absence of PDPN. Further analysis of WT and PDPN-deficient brains at different developmental time points and in the adult is needed to determine whether the morphological abnormalities precede hemorrhages and whether they are more pronounced near bleeding lesions.

To determine whether lack of PDPN causes impaired vascular integrity in adult mice, the inventor measured in vascular permeability in NE-Pdpn^{-/-} mice after a transient middle cerebral artery occlusion (tMCAO)-induced ischemia/reperfusion (see C1.3.2) using intravenously injected Evans blue. He observed that both control and injured hemispheres of WT brains exhibit no bleeding or leakage of Evans blue. In sharp contrast the injured side of NE-Pdpn^{-/-} mice developed bleeding with massive leakage of Evans blue (FIG. 4C). As NE-Pdpn−/− adult mice lack PDPN in astrocytes these preliminary data suggest the importance of astrocytic PDPN in vascular integrity upon vascular injury in the adult brain. Further experiments are needed to substantiate this result.

Whether PDPN in neuronal cells is required for integrity of nascent vessels in developing CNS. Identifying the cell types that express PDPN at different stages of CNS development is necessary to understand how PDPN functions in vascular integrity in the developing brain. Data show that E12.5 Cre8NE-Pdpn^{-/-} embryos develop the same CNS-specific bleeding phenotype observed in E12.5 Pdpn^{-/-} embryos (FIG. 2B), suggesting a function of PDPN in neural cells that supports vascular integrity. However, the inventor has not yet determined whether the brain hemorrhages in Cre8NE-Pdpn^{-/-} embryos, like Pdpn^{-/-} embryos, occur primarily between E10.5-14.5. To test this, the inventor will use timed-mating to generate E9.5-15.5 WT and Cre8NE-Pdpn^{-/-} embryos. Confocal immunostaining showed that E12.5 Cre8NE-Pdpn^{-/-} embryo lacked PDPN in all neural cells (data not shown). During early embryonic development (prior to E14), neuronal cells are the major neural cell type, as astrocytes are not differentiated at this stage in mice (Liu et al., 2002). Therefore, Cre8NE-Pdpn^{-/-} mice are an appropriate model to examine whether PDPN on neuronal cells is essential for vascular integrity. Complementarily, the inventor is generating mice that lack PDPN specifically in neuronal cells (NCPdpn^{-/-}) by breeding Pdpnf/f mice with a transgenic Cre line using the neuronal specific calmodulin-dependent kinase IIα promoter (JAX #005359) (Chen et al., 2001). The inventor will examine whether PDPN in NC-Pdpn^{-/-} mice is deleted specifically and sufficiently in neuronal cells during the early embryonic stage. If so, the inventor expects that both Cre8NE-Pdpn^{-/-} and NC-Pdpn^{-/-} embryos confer the embryonic bleeding phenotype observed in Pdpn^{-/-} embryos, which will indicate an essential role for PDPN in neuronal cells in imparting vascular integrity for nascent vessels in the developing CNS.

Role of PDPN astrocytes in maintaining vascular integrity in adult brain under pathological conditions. The inventor found that PDPN is selectively expressed in astrocytes surrounding larger vessels of adult brain (FIG. 3B). Astrocytes are differentiated in later stages of development and become mature after birth (Liu et al., 2002). In the adult, functional integrity of neurovasculature is maintained by the BBB and systemic hemostatic mechanisms under physiological conditions (Eichmann et al., 2005; Bautch and James, 2009; Tam and Watts, 2010). However, larger vessels such as arterioles have incomplete BBB properties (Abbott et al., 2006). The inventor asked whether PDPN in astrocyte endfeet activates platelets once integrity of a larger vessel is breached in order to prevent intracranial bleeding in the adult brain. To address this question, the inventor will use NE-Pdpn^{-/-} mice, a good model to determine the role of astrocyte PDPN in maintenance of vessel integrity mice because astrocytes are the only neural cell type expressing PDPN in the postnatal brain parenchyma (FIG. 3B).

Consistent with the inventor's hypothesis, over 90% (n=25) of NE-Pdpn^{-/-} mice developed spontaneous brain bleeding after birth (P0-3) (FIG. 2A). It is most likely that these were caused by CNS traumas occurring during delivery. The inventor will further investigate this by comparing WT and NE-Pdpn−/− neonates from either C-section surgery or natural birth. If bleeding is only observed in naturally delivered NE-Pdpn^{-/-} neonates, it will support that PDPN contributes significantly to maintaining neurovascular integrity under physical stress. The cerebral bleeding observed in NE-Pdpn^{-/-} neonates after birth is reminiscent of that associated with germinal matrixintraventricular hemorrhage (GHM-IVH) (roland and Hill, 2003). GMH-IVH develops in about 35% of human premature infants after birth and the cause is unknown. Identification of a novel role of PDPN in protecting newborns from brain bleeding may provide new insights into pathogenesis of diseases such as GMH-IVH

To determine the role of PDPN in astrocytes in protecting vascular integrity in the adult brain after injury, the inventor has have established a transient middle cerebral artery occlusion (tMCAO)-induced ischemia/reperfusion model that is widely accepted as a model for secondary hemorrhage in human patients with ischemic stroke (Fan et al., 2010). In pilot experiments, the inventor found that 7 out of 8 (88%) NEPdpn^{-/-} mice developed focal bleeding in the cerebral cortex and striatum regions after tMCAO. In contrast, only 2 out of 7 (29%) WT littermates exhibited brain bleeding (FIG. 5, and data not shown). These results support an important role for PDPN in astrocytes in maintaining vascular integrity in adult brain under pathological conditions such as stroke. The inventor will extend on this very promising result by including more mice in each group to improve statistical significance (8-16 weeks old, n=15 mice/group) and by including AC-Pdpn^{-/-} to substantiate the finding (8-16 weeks old, n=15 mice/group). In the pilot study, the inventor used 1.5 hr for ischemia and 42 hr for reperfusion. Under these conditions, two WT mouse developed limited brain bleeding, suggesting that the injury might have overcome the normal hemostatic threshold. In the proposed study, the inventor will therefore reduce the ischemia to 1 hr and reperfusion to 24 hr. In addition, he will include Magnetic Resonance Imaging (MRI) to detect early hemorrhage, monitor its progress, and quantify bleeding volume (every 10 h). This will be carried out in the MRI Core at The Oklahoma Medical Research Foundation.

Cerebral amyloid angiopathy (CAA) and hypertension are common risk factors for nontraumatic hemorrhagic stroke (Winkler et al., 2001). Transgenic mouse models of CAA develop spontaneous cerebral hemorrhage that closely resembles the human disease. In addition to the injury-induced model the inventor will test whether loss of PDPN in astrocytes causes spontaneous brain bleeding or exacerbates cerebral hemorrhage in the CAA model by breeding NE-Pdpn^{-/-} mice into a mouse line that models CAA (JAX #007027). Brain hemorrhage will be monitored in these mice every two weeks by MRI. Like hemorrhagic stroke, a hemorrhagic transformation of ischemic stroke is the most feared complication and often has devastating neurological consequences. Secondary hemorrhage may occur spontaneously within the core of a cerebral infarction or secondary to thrombolytic treatment. Based on strong preliminary results, the inventor predicts that NE-Pdpn^{-/-} or AC-Pdpn^{-/-} mice will be highly susceptible to the disease models relative to WT littermates. Strokes usually affect aged people. Thus, the inventor will compare young (8-12 weeks of age) and old (>1 yr old) WT and Pdpn mutants to test whether aged animals are more susceptible to the disease. If the inventor does not observe spontaneous bleeding in CAA/NE-Pdpn^{-/-} mice, they will be challenged with tMCAO.

Whether neuronal PDPN contributes to vascular integrity in the early developing brain. Pdpn^{-/-} and Cre8NE-Pdpn^{-/-} embryos exhibited brain bleeding that was most pronounced at E10-14 when BBB is not fully developed. This suggests an essential requirement for PDPN-CLEC-2-mediated platelet activation for vascular integrity in the early embryonic brain. Consistent with this, data show that E12.5 Pdpn^{-/-} brain vascular endothelial cells have abnormal structures that suggest impaired vascular integrity (FIGS. 4A-B). To test whether Pdpn^{-/-} nascent vessels are functionally impaired during this stage, vascular permeability assays will be performed using lysine-fixable Alexa555 Cad (Red, 0.9 kDa) or lysine-fixable dextran conjugated to FITC (FITC-dextran, green, 70 kDa, Invitrogen) to compare WT and Pdpn^{-/-} or Cre8NE-Pdpn^{-/-} embryos. Cad is a small molecule tracer and will be used to determine the general permeability of neurovasculature, while the larger molecular weight dextran will be used to detect breached vessels (Armulik et al., 2010). Cre8NE-Pdpn^{-/-} embryos at E12.5-14.5, which have functional circulation, will be given a trans-cardiac perfusion of these tracers (Daneman et al., 2010), respectively. WT and Pdpn^{-/-} embryos will be used as controls. Cre8NE-Pdpn−/− brain develops bleeding, which may cause localized extravasation of perfused tracers close to breached vessels. The inventor will focus the analyses on areas without bleeding. Based on strong preliminary data (FIGS. 2B and 4A), the inventor expects that both Pdpn^{-/-} and Cre8NE-Pdpn^{-/-} mice show increased accumulation of perfused tracers in the brain parenchyma and abnormal vascular ultrastructural morphology compared to their WT controls, which will indicate that PDPN in neuronal cells is critical for vascular integrity during early developmental stage.

Determine whether PDPN protects integrity of larger vessels in the CNS. In preliminary studies, the inventor detected PDPN-expressing astrocytes surrounding larger cerebral vessels with diameters ranging from 15-50 μm, but not capillaries (diameter less than 15 μm in general) in adult brain (FIG. 3B). Most of these larger vessels are round, with thick vessel-walls and a smaller inner diameter. These are morphological features of arterioles rather than veins. PDPN-deficient mice exhibit large, focal CNS hemorrhages rather than diffuse bleeding in the brain parenchyma, suggesting that the breaching occurs in high pressure, high volume vessels such as arterioles. The inventor therefore hypothesizes that PDPN is required for structural integrity of arterioles. To test this, the inventor will first determine whether astrocytic PDPN selectively surrounds larger vessels that are positive for arterial markers, such as D114 and Nrp1, using confocal microscopy. Compared to veins, arterioles should have stronger CD31 staining and be positive for specific arterial markers. The inventor expects that PDPN-expressing cells are associated with arterioles. If not, he will co-stain PDPN with venous markers such as Nrp2 and Coup-TF2. It is possible that PDPN is expressed in astroctyes surrounding both arterioles and venules. If so, it will suggest that PDPN protects both arterial and venous integrity in the brain.

To determine if large vessels that lack PDPN in adult mice are more sensitive to vascular injury, the inventor will use the Alexa Fluor-555 Cad or FITC-dextran described above. For perfusion, anesthetized NE-Pdpn^{-/-} or ACPdpn^{-/-} and WT littermate controls (8-16 weeks old, n=8-12 mice/group) will be perfused with the tracers into the left heart ventricle with or without tMCAO. This will be followed by perfusion with 4% PFA. Cryosections (30 μm) co-stained with arterial markers described above will be analyzed by conventional confocal microscopy to determine whether Pdpn mutant brains are more sensitive to leakage than WT brain with or without injury, and whether leakage occurs more frequently in arterioles. Alternatively, multiphoton confocal deep-tissue imaging of 100-500 μm thick brain sections will be used to enhance detection of arterial leakage. In a preliminary study, the inventor used this technique to measure FITC perfused brain vasculature and has established the feasibility of using this advanced imaging technique (LSM 7 MP two-photon confocal microscopy system, Carl Zeiss).

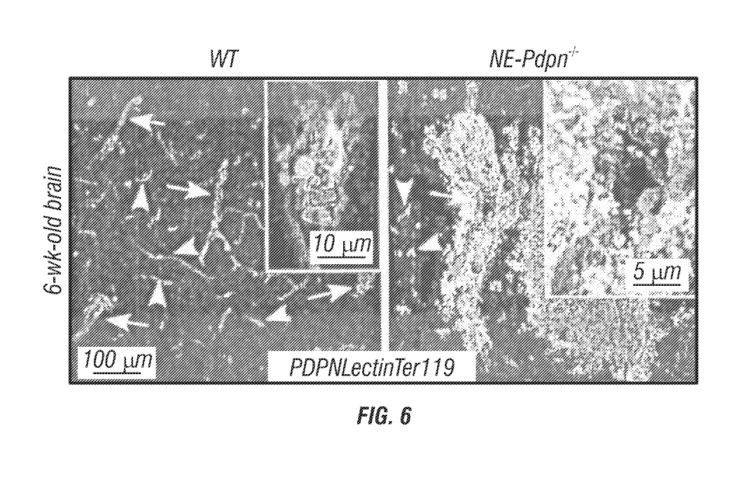

In these studies, the inventor found that bleeding in NE-Pdpn^{-/-} brain frequently occurred in larger vessels appeared to be arterioles (FIG. 6). Therefore, the inventor predicts that the proposed studies will permit definition of a role for astrocytic PDPN in controlling vessel leak and hemorrhage from arterioles in the adult brain after CNS injury. If leakages of perfused tracers occur in both arterioles and venules in Pdpn^{-/-} brain parenchyma, as opposed to arterioles, it will suggest that PDPN serves as a general mechanism protecting vascular integrity of larger vessels in the brain.

Clec2^{-/-} embryos develop CNS-specific hemorrhages reminiscent of Pdpn^{-/-} embryos. CLEC-2 is a transmembrane C type lectin-like receptor (Sobanov et al., 2001). It binds to glycan ligands and is the only known receptor for PDPN (Severin et al., 2011; Suzuki-Inoue et al., 2007). CLEC-2 is highly and selectively expressed on platelets, although it has also been reported to be on myeloid cells (Bertozzi et al., 2010; Kerrigan et al., 2009). CLEC-2 activates platelets through the non-receptor tyrosine kinase SYK and SLP-76 (Suzuki-Inoue et al., 2007). The inventor, as well as his collaborator, Dr. Mark Kahn, recently determined that interaction between PDPN on lymphatic endothelial cells and platelet CLEC-2 activates SYK/SLP-76 signaling in platelets, causing platelet aggregation that seals initial embryonic blood-lymphatic vascular connections at E12-13 (Fu et al., 2008; Bertozzi et al., 2010). These studies establish clearly that PDPN is a physiologically relevant ligand for CLEC-2 and reveal a novel platelet function in lymphatic vascular development. Whether PDPN-CLEC-2-mediated platelet activation plays a role other than controlling separation of blood and lymphatic vessels is unknown.

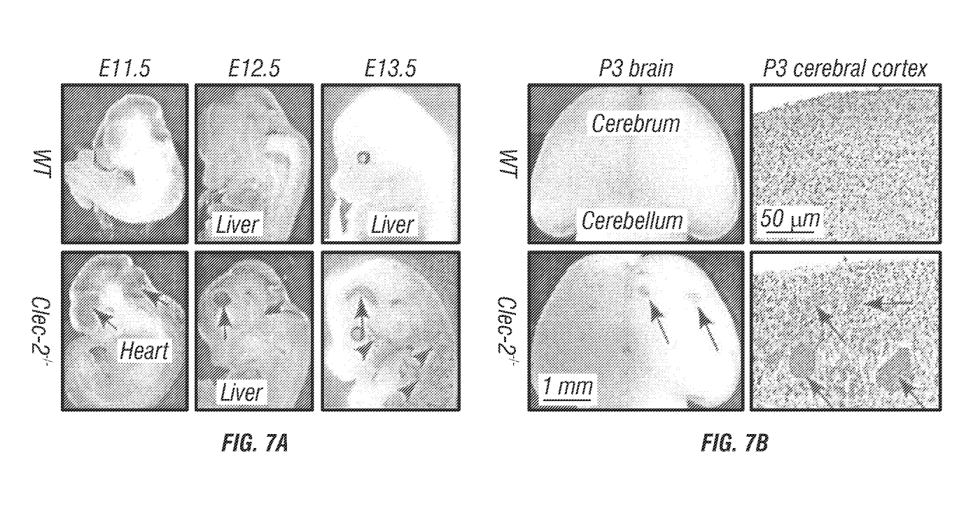

Remarkably, analysis of Clec2^{-/-} embryos revealed a virtually identical brain bleeding phenotype as Pdpn^{-/-} embryos (FIG. 7A). Like Pdpn^{-/-}, most Clec2^{-/-} die right after birth, and those that survive for a few days have brain hemorrhages similar to that found in Pdpn^{-/-} postnatal brains (FIG. 7B). This indicates an essential requirement for CLEC-2 in vascular integrity during development and after birth and suggests that PDPN and CLEC-2 act as partners for this function.

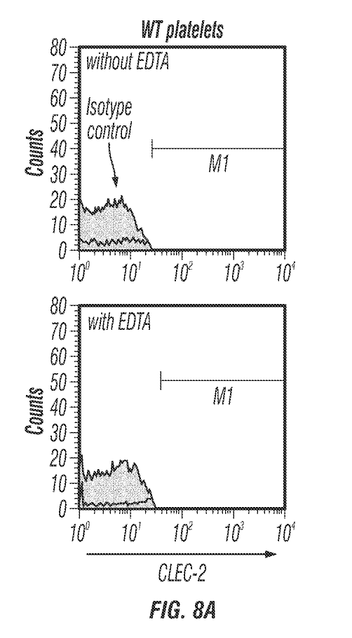

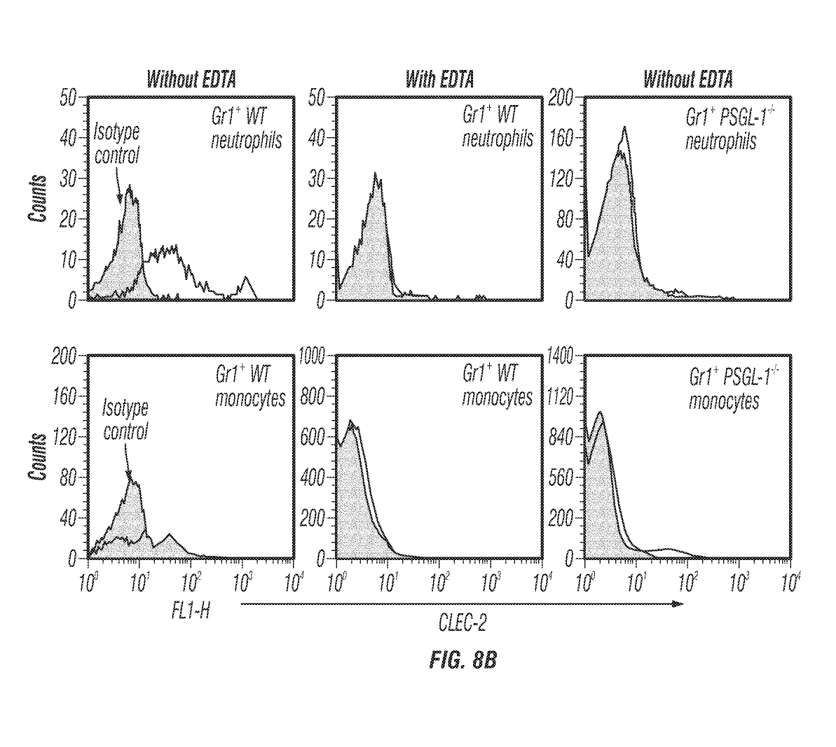

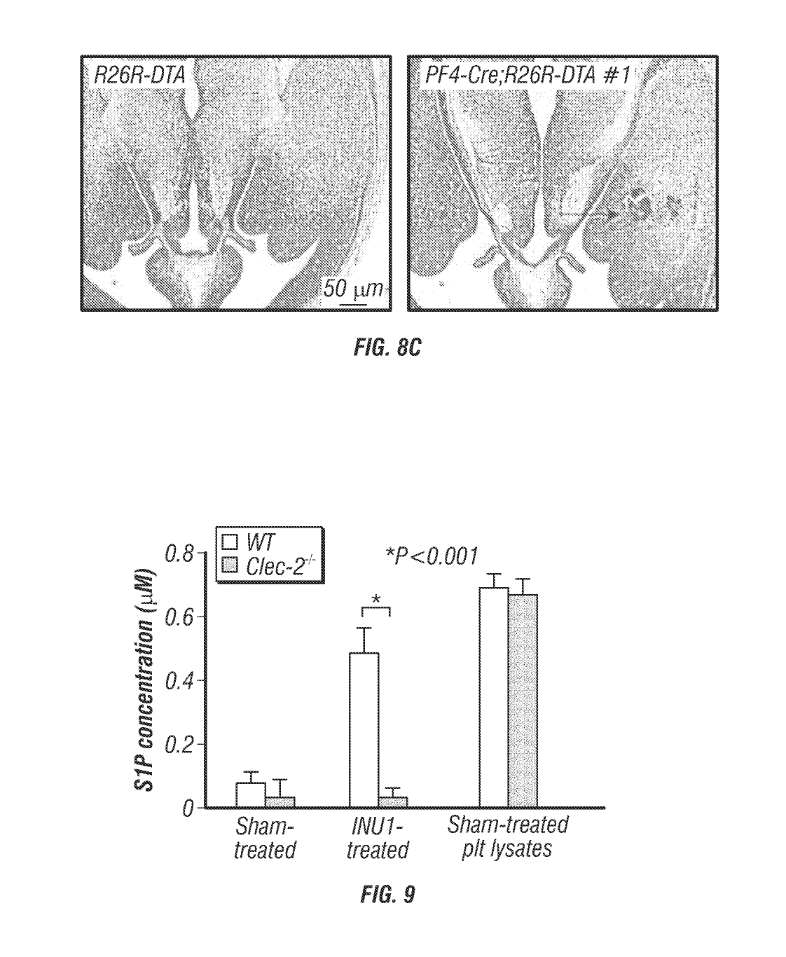

CLEC-2 is primarily expressed on platelets in peripheral blood. The primary hypothesis the inventor wishes to test is that PDPN in neural cells surrounding vessels binds and activates CLEC2 receptor on platelets. Published data suggest that CLEC2 is expressed on platelets as well as on neutrophils and monocytes (Sobanov et al., 2001; Kerrigan et al., 2009). The inventor has examined CLEC2 expression on peripheral blood cells from mice using two independent anti-CLEC2 antibodies (INU1 and 17D9) (May et al., 2009; Kerrigan et al., 2009) as well as PDPN-Fc fusion proteins (Bertozzi et al., 2010). Flow cytometry analysis showed that only platelets expressed CLEC2 (FIG. 8A, and data not shown). Neutrophils and monocytes incubated with EDTA or from P-selectin glycoprotein ligand 1-deficient mice (PSGL-1^{-/-}) did not have detectable CLEC2 (FIG. 8B). It can therefore be presumed that CLEC2 reported to be expressed by these cells arises from associated platelet microparticles.

Genetic loss of platelets results in bleeding in the CNS. Expression data reveal that CLEC2 is expressed exclusively by platelets, suggesting that the CNS hemorrhage observed in Clec-2^{-/-} embryos is platelet dependent. Since the brain bleeding phenotype has not been observed with other platelet deficiency states (especially β3 integrin deficiency) (Hodivala-Dilke et al., 1999), it is important to confirm that this is truly a platelet defect and is not due instead to an unexpected role of CLEC2 in other cell types. To clearly define the role of platelets in development of CNS hemorrhage the inventor generated PF4-Cre;R26RDTA mice that lack platelets entirely by crossing megakaryocyte/platelet-specific PF4-Cre with Cre-responsive ablator R26DTA mice (FIG. 8C) (Carramolino et al., 2010). In the PF4-Cre;R26RDTA mic, Cre-mediated recombination activated expression of the Diphtheria toxin subunit A (DTA), killing all megakaryocyte/platelets within 24 hours, but leaving neighboring cells unharmed (Wu et al., 2006).

E12.5 PF4-Cre;R26RDTA embryos exhibited bleeding in the CNS that mirrors that of both the Clec-2^{-/-} and Pdpn^{-/-} embryos. This confirms the requirement of platelets for vascular integrity in the CNS and also demonstrates that PF4-Cre can mediate deletion at the early developmental stage. Activation of CLEC-2 induces secretion of S1P from WT but not Clec-2^{-/-} platelets. The brain bleeding phenotype in mice lacking PDPN and CLEC-2 is reproduced in mice lacking platelets (FIG. 8C). Platelets are therefore a critical component for brain vascular integrity. Platelet adhesion, aggregate formation, granule secretion and/or procoagulant activity are essential for primary and secondary hemostasis (Furie and Furie, 2008).

In addition, brain bleeding is not a common symptom in patients that lack these platelet functions genetically, such as those with Bernard-Soulier syndrome (adhesion defect), Glanzmann thrombasthenia (no aggregation), gray platelet syndrome (a granule deficiency), and Chediak-Higashi syndrome (dense granule deficiency) (Nurden and Nurden, 2011). This suggests that platelet function mediated by PDPN-CLCE-2 signaling that is distinct from or, at least, in addition to aggregation and granule release is required for vascular integrity. One strong candidate for this platelet function is S1P, which has attracted much attention for its role as a key regulator of vascular integrity, interacting with its G protein-coupled receptors on various target cells such as endothelial cells (Camerer et al., 2009). S1P is abundantly stored in the cytosol of platelets and its release occurs independently from degranulation (Ulrych et al., 2011). Significantly, mice lacking S1P or its receptors, S1P1 or 3, which are highly expressed on vascular endothelial or mural cells, have a brain bleeding phenotype that is reminiscent of the one seen in Pdpn^{-/-} or Clec-2^{-/-} brains (Kono et al., 2004; Mizugishi et al., 2005). These results support the hypothesis that S1P released from the cytosol of platelets activated by PDPN-CLEC-2 signaling plays an essential role in vascular integrity in the brain.

Platelets generate and store high amounts of S1P, which is released upon stimulation with activators of protein kinase C, such as thrombin (Camerer et al., 2009; Ulrych et al., 2011). Whether PDPN-CLEC-2 signaling-mediated platelet activation results in S1P release is unknown. To address, this inventor performed a pilot experiment in which platelets from WT and Clec-2^{-/-} mice were isolated and stimulated with an anti-CLEC-2 antibody, INU1 (May et al., 2009), which is known to activate CLEC-2. S1P released into supernatants was measured using an S1P ELISA kit (Echelon Biosciences). Significantly, INU1 treatment resulted in a release of S1P from WT platelets. However, INU1 did not induce S1P release from Clec-2^{-/-} platelets (FIG. 9), supporting that CLEC-2 signaling is required for S1P secretion. This important preliminary result revealed a novel pathway of S1P release and provided a proof of principle for this hypothesis.

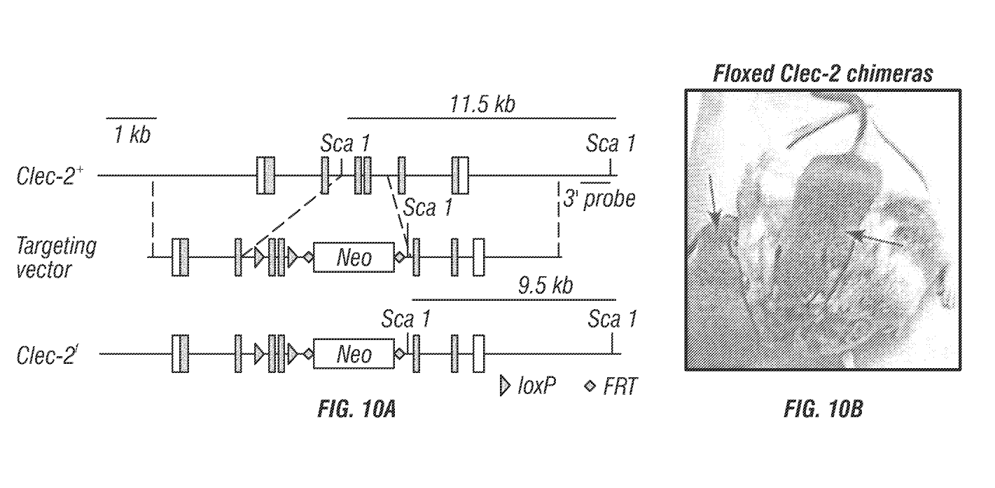

Whether platelet CLEC-2 is required for vascular integrity in the CNS. Although the data above demonstrate that CLEC-2 is primarily expressed on platelets in peripheral blood and platelets are required for vascular integrity in the brain (FIGS. 8A-C), to rule out unexpected role for CLEC-2 in other cell types (e.g., endothelial cells) and definitively address whether platelet CLEC-2 is required for vascular integrity in the brain, the inventor is developing a floxed Clec-2 conditional mouse line (Clec-2f/f) (FIG. 10). Platelet-specific Clec-2-deficient mice (Plt-Clec-2^{-/-}) will be obtained by breeding Clec-2f/f with PF4-Cre mice that allow deletion specifically in megakaryocytes and platelets (Tiedt et al., 2007). Timed mating will be used to generate Plt-Clec-2^{-/-} embryos to determine whether platelet CLEC-2 is required for vascular integrity in the developing CNS. Brain injury models described C1.3.2 will be used to test whether adult Plt-Clec-2^{-/-} mice are more susceptible to brain bleeding than WT littermate controls. It is possible that Plt-Clec-2^{-/-} mice will exhibit high neonatal lethality similar to Clec-2−/− mice. If this occurs, the inventor will develop bone marrow chimeras by transplanting E15Plt-Clec-2^{-/-} fetal liver cells into lethally irradiated WT recipients (Clec-2^{-/-} chimera), which is an established technique in this lab (Fu et al., 2008; Xia et al., 2004). The inventor expects that platelet CLEC-2 is required for vascular integrity in the developing and mature CNS.

Whether platelet activation by PDPN-CLEC-2 signaling is required for vascular integrity in the CNS. These studies indicate that platelets are required for vascular integrity in the brain. However, the inventor has not determined whether platelet activation by PDPN-CLEC-2 signaling is required for this. Platelet activation by interaction of PDPN with CLEC-2 requires the tyrosine kinase SYK. If CLEC-2 signaling-mediated platelet activation is required to maintain vascular integrity, mice lacking platelet SYK (Plt-Syk^{-/-}) should exhibit a brain bleeding phenotype similar to that of mice lacking PDPN or CLEC-2. The inventor will use Plt-Syk^{-/-} mice to test this hypothesis (Severin et al., 2011; Sebzda et al., 2006). The inventor expects that Plt-Syk^{-/-} mice will exhibit the same bleeding phenotype as PDPN or CLEC-2 mutants. Results from this study will be analyzed in conjunction with results from Plt-Clec-2^{-/-} mice described above. If both Plt-Clec-2^{-/-} and Plt-Syk^{-/-} mice exhibit the same brain bleeding phenotype as PDPN mutant mice it will provide strong support for the hypothesis that platelet activation mediated by the PDPN-CLEC-2 signaling pathway is required for CNS-specific vascular integrity. If Plt-Clec-2^{-/-}, but not Plt-Syk^{-/-}, shows the phenotype it will suggest that PDPN-CLEC-2 uses an alternative signaling pathway. In this event the inventor will work with a platelet signaling expert, to explore alternative signaling pathways.

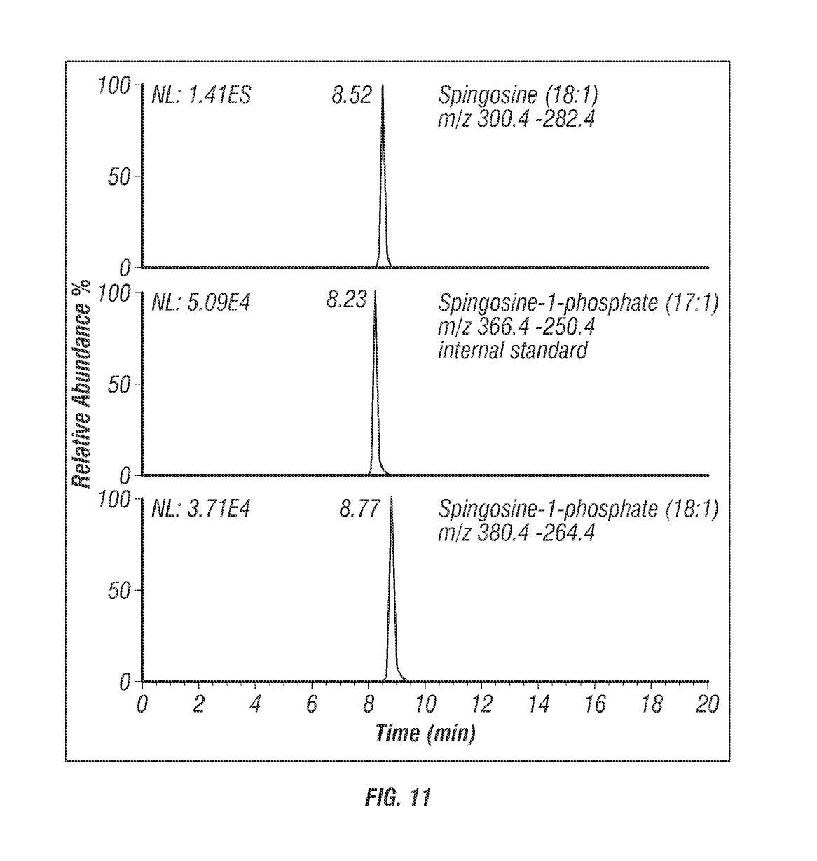

The role in vascular integrity of S1P released after PDPN-CLEC-2-dependent platelet activation. The analysis shows that platelet function other than aggregation and granule release is required for PDPN-CLEC-2-mediated vascular integrity. The inventor hypothesizes that platelet CLEC2 activation by tissue PDPN takes place after extravasation of blood and that PDPN-CLEC-2-activated platelets increase the local concentration of S1P to maintain vascular integrity (FIG. 11). S1P in the tissue may function by interacting with its receptors such as S1P1-3 on mural cells or the ablumenal side of endothelial cells (Kono et al., 2004). Supporting this, the inventor observed in preliminary analyses an abnormal morphology of endothelial cells and impaired integrity of brain vessels (FIG. 5). Significantly, these data indicate that activation of CLEC-2 induces release of S1P from platelets (FIG. 9). These data provided sufficient evidence for the inventor to test the hypothesis.

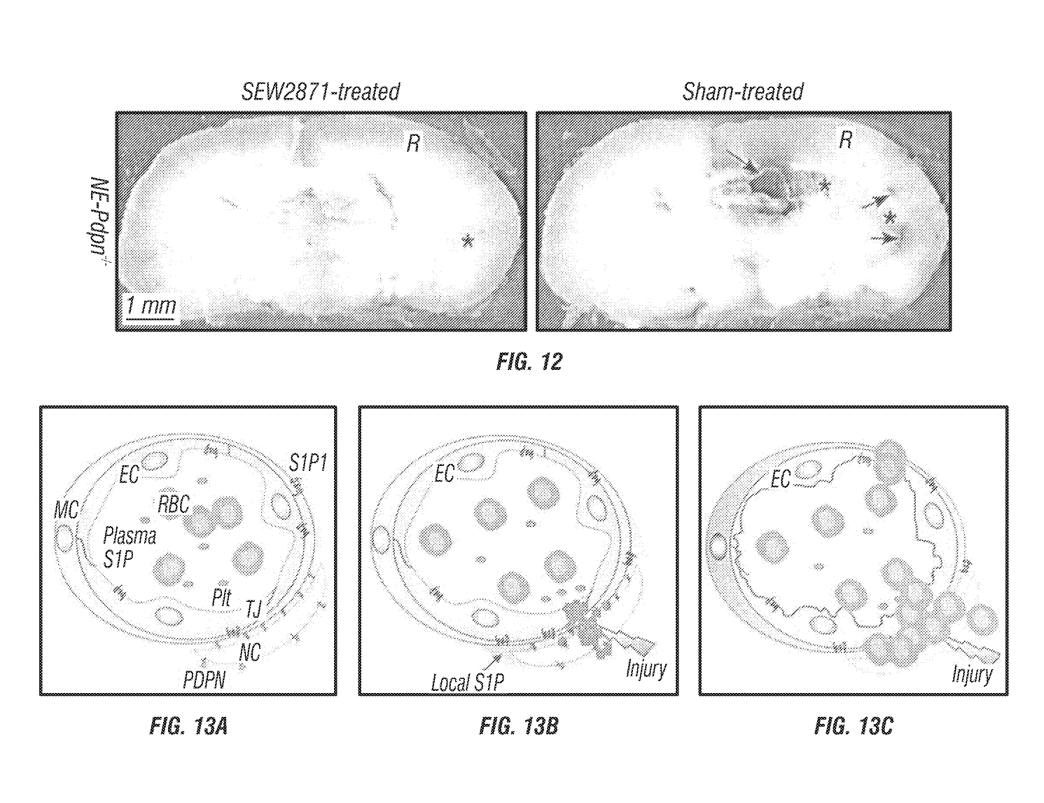

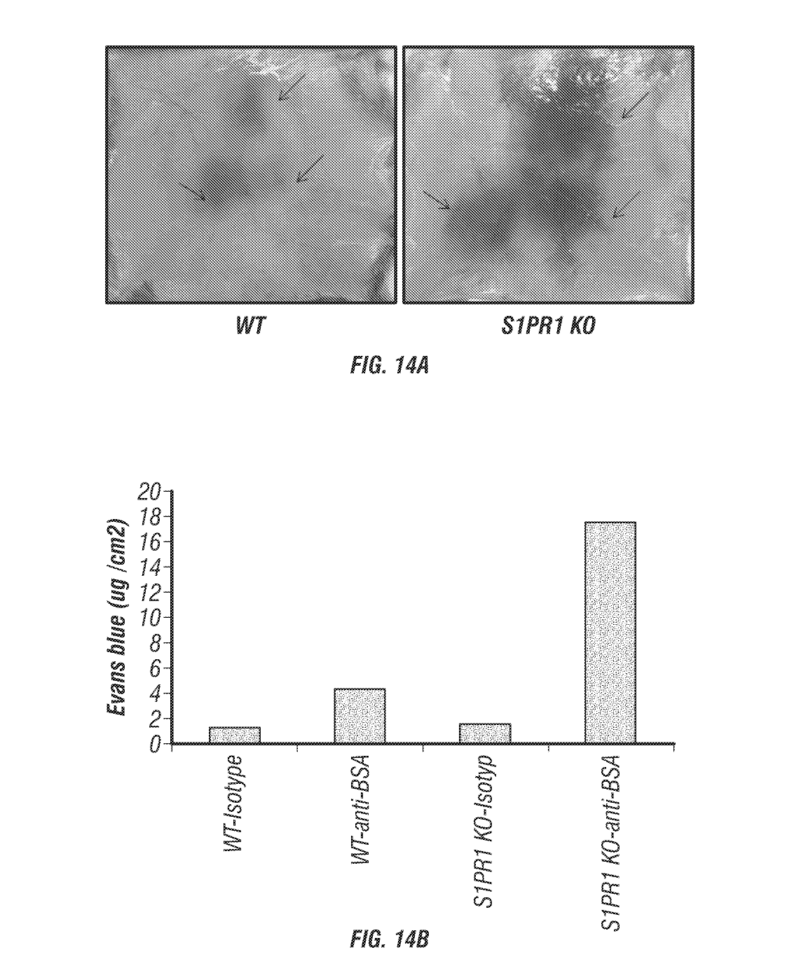

Determine whether PDPN-CLEC-2 signaling results in S1P release from activated platelets. In experiments, the inventor determined that ligation of CLEC-2 with the anti-CLEC-2 antibody INU1 induces S11) release from platelets measured by ELISA (FIG. 9). This important result revealed a novel pathway of S1P release and provided a proof of principle for the inventor's hypothesis. He will repeat this experiment with additional samples, as well as using recombinant murine PDPN that is being generated in the inventor's lab as a natural ligand for CLEC-2 activation, and by including Syk-deficient platelets, to test whether this is PDPN/CLEC-2/Syk signaling dependent. The LISA result will be consolidated with a complementary approach for S1P detection using mass spectrometry (FIG. 12) that the inventor has developed with help from Dr. Michael Kinter, an expert in mass spectrometry, at the inventor's institution. Using these complementary methods, the inventor will also explore the possibility to measure S1P in the brain tissue to determine whether lack of PDPN-CLEC-2 decreases local S1P concentration. He will start with tissue lysates from WT and NE-Pdpn^{-/-} adult brain after vascular perfusion with saline to remove plasma S1P after tMCAO. Non-bleeding brain tissues will be dissected and assayed for S1P to rule out contamination of plasma SIP. If successful, the inventor will include Plt-Clec-2^{-/-} and Plt-Syk mice. Based on preliminary data, the inventor expects that PDPN-CLEC-2 signaling induces S1P release from platelets and that SIP in WT adult brain after vascular injury will be higher than that in NE-Pdpn^{-/-} brain. This result would support the inventor's hypothesis that PDPN-CLEC-2-mediated release of S1P is required for vascular integrity to prevent vascular leakage and bleeding.

Test whether S1P1 specific agonist ameliorates brain bleeding from PDPN-CLEC-2 signaling-deficient mice. The S1P receptor S1P1 is highly expressed on vascular endothelial cells (Kono et al., 2004). Mice lacking S1P1 develop brain bleeding during embryonic development that is similar to mice lacking PDPN or CLEC-2. If PDPN-CLEC-2-mediated release of S1P from activated platelets is essential for vascular endothelial function in the brain, S1P1 agonist should rescue/ameliorate the brain bleeding phenotype. To test this, the inventor performed a pilot experiment and found that the S1P1 specific agonist SEW2871 (100 mg/kg body weight, i.p. in sunflower oil, Cayman Chemical, Michigan) ameliorated brain bleeding and vascular leakage in NE-Pdpn^{-/-} mice after tMCAO (1.5 hr ischemia and 24 hr reperfusion, n=2/group) (FIGS. 13A-C). Although preliminary, this promising result provides a proof of principle and establishes the feasibility of this approach. The inventor will incorporate additional animals into this study. S1P-mediated signaling is dynamic (Camerer et al., 2009). The inventor will therefore test different dosages of SEW2871. The inventor will also administer the agonist prior to or after tMCAO to test whether the treatment has preventive or therapeutic effects. Cerebral edema as a result of increased vascular leakage is a devastating complication of stroke or brain trauma. The inventor observed in preliminary experiments that NE-Pdpn^{-/-} mice had significantly increased vascular permeability after tMCAOrelative to WT mice, suggesting that PDPN in astrocytes is required to protect vascular integrity after injury. The inventor will test whether SEW2871 reduces cerebral vascular leakage and edema after tMCAO. For this purpose, the inventor will shorten the ischemia (1 hr) and reperfusion time (Furie and Furie, 2008; Fu et al., 2008; Bertozzi et al., 2010; May et al., 2009; Severin et al., 2011) in the tMCAO model so that NE-Pdpn^{-/-} mice will have increased vascular permeability but not bleeding. Cerebral vascular permeability/edema will be detected by intravenous injection of Evans blue and the wet/dry ratio of the brains. Vascular integrity will be analyzed by confocal imaging and/or TEM as described in C1.3.3. The inventor will use a S1P1 specific antagonist (VPC23019, R&D, Minneapolis) to test whether SEW2871 specifically functions on S1P1, which is highly expressed on endothelial cells, to protect vascular integrity.

Test whether mice lacking platelet SIP exhibit a brain bleeding phenotype. SIP is synthesized by phosphorylation of sphingosine through the action of sphingosine kinase (SphK) (Camerer et al., 2009). There are two isoforms of SphK (SphK1 and SphK2) in mammals. Mice lacking either SphK1 or SphK2 exhibit no significant abnormalities, indicating that the enzymes have overlapping function. However, mice lacking both SphK1 and SphK2 develop brain bleeding around E11.5 that is reminiscent of the Pdpn−/− and the Clec-2−/− embryos (Mizugishi et al., 2005).

These results indicate that S1P is essential for vascular integrity in the brain. Many cell types, including platelets, produce S1P. The inventor will determine whether S1P from platelets plays a critical role in vascular integrity in the brain. To test this, the inventor will obtain a mouse line with one conditional Sphk1 allele and one null Sphk1 allele in an Sphk2-null background from a consultant and breed this line with a PF4-Cre tg (currently maintained in the inventor's lab) to generate mice lacking S1P specifically in megakaryocytes/platelets (Plt-SphK1/SphK2^{-/}). The inventor expects that Plt-SphK1/SphK2^{-/-} mice will recapitulate the brain bleeding phenotype of SphK1 and SphK2 global double knockout. If so, it will support the importance of platelet S1P in vascular integrity in the CNS. If not, it will suggest either that S1P from platelets is not sufficient or S1P from other cell types is essential for this function. Either result will lead the inventor to design further experiments to identify the source of the S1P required for regulating vascular integrity.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
U.S. Pat. No. 4,256,108
U.S. Pat. No. 4,166,452
U.S. Pat. No. 4,265,874
Abbott et al., Nat. Rev. Neurosci., 7(1):41-53, 2006.
Armulik et al. Nature, 468(7323):557-561, 2010.
Bautch and James, Cell Adh Migr., 3(2):199-204, 2009.
Bertozzi et al., Blood, 116(4):661-670, 2010.
Borok et al. Am. J. Physiol., 275(1Pt 1):L155-164, 1998.
Camerer et al., J. Clin. Invest., 119(7):1871-1879, 2009
Carramolino et al. Circ., Res., 106(7):1197-1201, 2010.
Chen et al., Nat. Genet., 27(3):327-331, 2001.
Daneman et al., Nature, 468(7323):562-566, 2010.
Eichmann et al., Curr. Opin. Neurobiol., 15(1):108-15, 2005.
Fan et al., Ann. Neurol., 67(4):488-497, 2010.
Fu et al., J. Clin. Invest., 118(11):3725-3737, 2008.
Furie and Furie, N. Engl. J. Med., 359(9):938-949, 2008.
Goodman & Gilman's “The Pharmacological Basis of Therapeutics,
Hasegawa et al., Stroke, 41(2):368-374, 2010.
Hodivala-Dilke et al., J. Clin. Invest., 1103(2):229-238, 1999.
Jain, Nat. Med., 9(6):685-693, 2003.
Juarez et al., Blood, 119(3):707-716, 2012.
Kato et al., J. Biol. Chem., 278(51):51599-51605, 2003.
Kerrigan et al., J. Immunol., 182(7):4150-4157, 2009.
Kono et al., J. Biol. Chem., 279(28):29367-29373, 2004.
Kotani et al. J. Neurosci. Res., 73(5):603-613, 2003.
Liu et al. Glia., 40(1):25-43, 2002.
May et al., Blood, 114(16):3464-3472, 2009.
Mizugishi et al., Molec. Cell. Biol., 25(24):11113-11121, 2005.
Murakami and Simons, J. Mol. Med., 87(6):571-582, 2009.
Nurden and Nurden, J. Thromb. Haemost., 9(Suppl 1):76-91, 2011.
Petersen et al. Nature, 419(6910):929-934, 2002.
Physicians Desk Reference,
Ramirez et al. Dev. Biol., 256(1):61-72, 2003.
Remington's Pharmaceutical Sciences
Roland and Hill, Neurol. Clin., 21(4):833-851, 2003.
Schacht et al. Embo. J., 22(14):3546-3556, 2003.
Sebzda et al., Developmental Cell, 11(3):349-361, 2006.
Severin et al., J. Biol. Chem., 286(6):4107-4116, 2011.
Sobanov et al., EP J. Immunol., 31(12):3493-3503, 2001.
Suzuki-Inoue et al., J Biol. Chem., 282(36):25993-26001, 2007.
Tam and Watts, Annu. Rev. Neurosci., 33:379-408, 2010.
The Merck Index, Thirteenth Edition
Tiedt et al., Blood, 109(4):1503-1506, 2007.
Tronche et al., Nat. Genet., 23(1):99-103, 1999.
Ulrych et al., J. Thromb. Haemost., 9(4):790-798, 2011.
Winkler et al., J. Neurosci., 21(5):1619-1627, 2001.
Wu et al., Development, 133(4):581-590, 2006.
Xia et al., Blood, 104(10):3091-3096, 2004.

What is claimed is:

1. A method of inhibiting vascular leakage in central nervous system (CNS) tissue in a subject comprising administering to said subject an agonist or mimic of podoplanin (PDPN)/C-type lectin-like receptor 2 (CLEC-2) signaling.

2. The method of claim 1, wherein said CNS tissue is brain tissue.

3. The method of claim 1, wherein said vascular leakage is due to trauma.

4. The method of claim 1, wherein said vascular leakage is due to stroke.

5. The method of claim 1, wherein said agonist or mimic is administered systemically.

6. The method of claim 1, wherein said agonist or mimic is delivered to said CNS tissue.

7. The method of claim 1, wherein said agonist or mimic is delivered multiple times.

8. The method of claim 1, wherein said agonist or mimic is delivered continuously over a period of time exceeding 1 hour.

9. The method of claim 1, wherein said agonist or mimic is delivered within 2 hours of the initiation of vascular leakage.

10. The method of claim 1, wherein said subject is a human.

11. The method of claim 1, wherein said agonist is a soluble form of PDPN.

12. The method of claim 1, wherein said mimic is a PDPN mimic.

13. The method of claim 1, wherein said agonist is a downstream effector that results from PDPN/CLEC-2 signaling.

14. The method of claim 13, wherein said effector is S1P.

15. The method of claim 1, wherein said mimic is a mimic of a downstream effector that results from PDPN/CLEC-2 signaling.

16. The method of claim 15, wherein said effector mimic is an S1PR1 agonist.

17. The method of 1, further comprising administering to said subject a second agent that inhibits vascular leakage.

18. The method of claim 17, wherein said second agent is administered at the same time as said agonist or mimic.

19. The method of claim 17, wherein said second agent is administered before or after said agonist or mimic.

20. The method of claim 17, wherein said second agent is administered on an alternating basis with said agonist or mimic.

21. A method of promoting vascular integrity in central nervous system (CNS) tissue in a subject comprising administering to said subject an agonist or mimic of podoplanin (PDPN)/C-type lectin-like receptor 2 (CLEC-2) signaling.

22. The method of claim 21, wherein said agonist or mimic is administered systemically.

23. The method of claim 21, wherein said agonist or mimic is delivered to said CNS tissue.

24. The method of claim 21, wherein said subject is a human.

25. The method of claim 21, wherein said agonist is a soluble form of PDPN.

26. The method of claim 21, wherein said mimic is a PDPN mimic.

27. The method of claim 21, wherein said agonist is a downstream effector that results from PDPN/CLEC-2 signaling.

28. The method of claim 27, wherein said effector is S1P.

29. The method of claim 21, wherein said mimic is a mimic of a downstream effector that results from PDPN/CLEC-2 signaling.

30. The method of claim 29, wherein said effector mimic is an S1PR1 agonist.

31. A method of inhibiting platelet activation in central nervous system (CNS) tissue in a subject comprising administering to said subject an antagonist of podoplanin (PDPN)/C-type lectin-like receptor 2 (CLEC-2) signaling.

32. The method of claim 31, wherein said CNS tissue is brain tissue.

33. The method of claim 31, wherein said platelet activation leads to thrombosis.

34. The method of claim 31, wherein said platelet activation leads to stroke.

35. The method of claim 31, wherein said antagonist is administered systemically.

36. The method of claim 31, wherein said antagonist is delivered to said CNS tissue.

37. The method of claim 31, wherein said antagonist is delivered multiple times.

38. The method of claim 31, wherein said subject is a human.

39. The method of claim 31, wherein said antagonist is an antibody that binds selectively to PDPN, CLEC-2 or an inactive fragment of PDPN to CLEC-2 that interferes with the binding of PDPN to CLEC-2.

40. The method of claim 1, wherein said antagonist is an siRNA that inhibits production of PDPN or CLEC-2.
